(19) United States
(12) Patent Application Publication
(10) Pub. No.: US 2013/0035736 A1
(43) Pub. Date: Feb. 7, 2013

(54) SYSTEMS AND METHODS FOR CONTROLLING PAIRED PACING BASED ON PATIENT ACTIVITY FOR USE WITH AN IMPLANTABLE MEDICAL DEVICE

(71) Applicant: Steve Koh, South Pasadena, CA (US)

(72) Inventor: Steve Koh, South Pasadena, CA (US)

(73) Assignee: PACESETTER, INC., Sylmar, CA (US)

(21) Appl. No.: 13/196,763

(22) Filed: Aug. 2, 2011

(51) Int. Cl. A61N 1/368

(52) U.S. Cl. 607/18; 607/19

(57) ABSTRACT

Techniques are provided for use with implantable medical devices equipped to deliver paired postextrasystolic potentiation (PESP) pacing to control the paired pacing rate based on changes in patient activity. In one example, the current activity level of the patient is detected during paired pacing using an accelerometer. The cardiac output level needed to maintain the current activity level of the patient is determined with reference to pre-stored lookup tables relating activity levels with corresponding minimum necessary cardiac output levels for the particular patient. The minimum paired pacing rate sufficient to achieve the cardiac output level is then determined based, e.g., on stroke volume derived from cardiogenic impedance signals. Paired pacing is then delivered at the minimum paired pacing rate sufficient to achieve the needed cardiac output, thereby assuring that the paired pacing rate is sufficient to meet the current physiological demands of the patient without consuming too much oxygen.

### FIELD OF THE INVENTION

The invention generally relates to implantable cardiac stimulation devices such as pacemakers and implantable cardioverter-defibrillators (ICDs) and, in particular, to techniques for controlling paired pacing achieved via postextrasystolic potentiation (PESP).

### BACKGROUND OF THE INVENTION

PESP therapy is a pacing therapy wherein extra stimulation pulses are delivered by a pacemaker or other suitable device during (or sometimes just outside of) a relative myocardial refractory period following paced or intrinsic depolarization. The extra PESP stimulus causes the heart muscle to depolarize a second time but does not cause significant contraction of the muscle. The second depolarization acts on the sarcoplasmic reticulum to release an additional bolus of calcium. It is generally believed that the additional intracellular calcium ions provide for increased contractility. Another consequence of the extra stimulus provided during the relative refractory period is to extend the overall refractory interval, which slows the heart and allows the pacemaker to control the heart rate. During actual delivery of PESP pulses, as with all stimulation pulses, the pacemaker blanks or blocks its sensing channels so as not to misinterpret the electrical stimulus as being an intrinsic electrical signal (i.e. an electrical signal arising from the myocardial tissue.)

Note that, following a paced or intrinsic depolarization, pacemakers typically track a refractory interval that includes both an absolute refractory period and a subsequent relative refractory period. During the absolute refractory period, a second myocardial depolarization cannot be triggered, regardless of the amplitude of extra stimulus, because the myocardial tissue is not susceptible to further electrical stimulus at that time. Hence, PESP pulses are not delivered during the absolute refractory period. During the subsequent relative refractory period, a second depolarization can be triggered with a sufficiently large stimulation pulse but not with a pulse of otherwise normal pulse amplitude. Accordingly, PESP pulses are typically delivered during the relative refractory period (or sometimes just beyond it) using a stimulation pulse of nominal pulse amplitude to trigger depolarization without contraction.

PESP can be implemented in accordance with either “paired pacing” or “coupled pacing” techniques. With paired pacing, the additional PESP pulse is delivered following a paced depolarization. With coupled pacing, the additional stimulation is delivered following an intrinsic depolarization. Paired and coupled pacing techniques are discussed in U.S. Published Patent Application No. 2010/0094371 of Bornzin et al., entitled “Systems and Methods for Paired/Coupled Pacing” and in U.S. patent application Ser. No. 11/929,719, also of Bornzin et al., filed Oct. 30, 2007, entitled “Systems and Methods for Paired/Coupled Pacing and Dynamic Overdrive/Underdrive Pacing.”

PESP may be used to enhance cardiac resynchronization therapy (CRT) by increasing contractility beyond what is typically achieved by merely restoring synchrony. PESP may be used to slow the ventricles during atrial fibrillation (AF) because PESP tends to prolong the refractory interval. That is, the additional depolarization during the relative refractory period caused by the PESP pulse has the effect of extending the overall refractory interval. The longer refractory interval acts to block the conduction of rapid atrial impulses associated with AF. PESP thus can provide for rate control during AF. A secondary benefit may be enhanced contractility for patients with AF and heart failure. Further, PESP may be used to treat patients with low ejection fraction (EF) and narrow QRS heart failure (i.e. a form of heart failure wherein the electrical signals associated with ventricular depolarization (QRS complexes) are shorter than usual.) PESP may be used to treat their cardiac insufficiency. Still further, PESP may be used to treat heart failure with preserved EF. Patients with heart failure with preserved EF can benefit because PESP enhances the rate of relaxation.

Hence, PESP can be particularly useful in patients with poor EF, which might not be corrected by standard CRT or medicinal (Rx) therapy. Possible candidates include: (1) CRT non-responders (which currently represent ˜30% of CRT device patients); (2) patients with narrow QRS (<120 ms) currently not indicated for standard CRT; (3) diastolic heart failure (HF) patients not indicated for device therapy; and (4) AF patients with rapidly conducted heart beats. These and other clinical problems may be helped by the benefits of PESP, which can include: (a) reduce rate to allow for longer filling time; (b) increased EF; and (3) reduced rate during rapidly conducted AF.

One issue to be addressed with PESP is how best to determine the pacing rate for paired pacing. This is important since the paired pacing rate will determine cardiac output (CO). If the device paces too slowly during paired pacing, then overall CO might not meet the physiological demand of the patient due to on-going patient activity. In this regard, if the rate is too low, then even though the PESP-driven stroke volume (SV) might be higher than that of normal sinus rhythm, the CO still might not meet the physiological demands of the patient, particularly during activity that is more strenuous. (For coupled pacing, where PESP pulses are coupled to intrinsic depolarizations, the heart beats at its intrinsic rate and hence the device need not determine a separate PESP rate.) Another issue is that paired pacing, if performed at a rate that is too high, may consume too much oxygen, which can be a problem particularly within heart failure patients. For these and other reasons, at least some patients who are candidates for paired pacing do not currently receive paired pacing.

Accordingly, it would be desirable to provide techniques for addressing these and other concerns and it is to this end that aspects of the invention are generally directed.

### SUMMARY OF THE INVENTION

In an exemplary embodiment, a method is provided for use with an implantable cardiac stimulation device equipped to deliver paired pacing via PESP. In accordance with an exemplary method, a current activity level of the patient is detected during paired pacing. The cardiac output (CO) level needed to maintain the patient's current activity level is determined during paired pacing with reference, e.g., to pre-stored lookup tables relating activity levels with corresponding minimum necessary CO levels for the particular patient. A minimum or target paired pacing rate sufficient to achieve the CO level is then determined based, e.g., on stroke volume (SV) derived from cardiogenic impedance (also referred to as intra-cardiac impedance). Paired pacing is then delivered at the determined paired pacing rate, thereby assuring that the paired rate is sufficient to meet the current physiological demands of the patient but is not set higher than needed.

In an exemplary embodiment where the implanted device is a pacemaker, ICD or CRT device, the current activity level of the patient is detected during paired pacing using an accelerometer. The accelerometer output value is applied to a pre-stored lookup table that relates accelerometer values to corresponding CO levels for the patient to thereby readout a target CO value, which represents the minimum necessary CO needed to meet the physiological demand within the patient at the current activity level. To determine the paired rate needed to achieve the target CO level, the device estimates the current SV within the patient from a cardiogenic impedance signal (Z(i)) based, for example, on an examination of delta (Z(i)) or area (Z(i)). Once the current SV has been ascertained, the device divides CO by SV to determine the paired pacing rate needed to achieve the target CO level to meet the current physiological demands of the patient. The procedure is repeated as needed to update the paired rate based on changes in activity, stroke volume or other factors. For example, the procedure might be repeated every few cardiac cycles or might be repeated whenever a significant change is detected in activity level, stroke volume or other parameters.

An initial calibration procedure may be performed to determine the relationship between activity level and corresponding cardiac output levels for the particular patient for use in populating the lookup table. The initialization procedure is performed without any on-going paired pacing (or other pacing.) In one example, the intrinsic heart rate of the patient is tracked while parameters representative of cardiogenic impedance (Z) are detected and while patient activity is tracked using an accelerometer. Stroke volume is estimated from cardiogenic impedance, then CO is estimated by multiplying SV by the intrinsic heart rate. The estimated CO is stored in the lookup table along with the corresponding output value (XLS) from the accelerometer to thereby relate CO to activity within the patient at a particular activity level. This procedure is repeated for various activity levels throughout a range of patient activity to thereby populate the lookup table. For example, the patient might be instructed to exercise at each of various activity levels under clinician supervision to thereby generate a set of CO values and corresponding accelerometer output values for populating the lookup table. The initialization/calibration procedure can be performed by the device itself, if so equipped, or by an external system such as a device programmer operated under clinician supervision.

Rather than using a lookup table, other equivalent means for relating cardiac output to patient activity can instead be employed, such as computational devices that use functional equations to relate CO to patient activity. If so, the parameters defining those equations can be ascertained during the initialization procedure based on linear regression or other suitable techniques. Also, it should be understood that whenever cardiogenic/intracardiac impedance is detected, other suitable electrical parameters could instead be employed, such as admittance, conductance or immittance.

System and method implementations are described herein.

### BRIEF DESCRIPTION OF THE DRAWINGS

Features and advantages of the described implementations can be more readily understood by reference to the following description taken in conjunction with the accompanying drawings.

FIG. 1 illustrates components of an implantable medical system having a pacemaker, ICD or CRT device equipped to control PESP paired pacing based on patient activity in accordance with an exemplary embodiment of the invention;

FIG. 2 summarizes a general technique for controlling PESP paired pacing based on patient activity that may be performed by the system of FIG. 1;

FIG. 3 is a flowchart illustrating a preliminary calibration/initialization procedure for determining the relationship between CO and activity levels for the patient (e.g. for generating a suitable lookup table relating CO and patient activity) for subsequent use with the general technique of FIG. 2;

FIG. 4 illustrates exemplary signal components processed by the calibration/initialization method of FIG. 3 to generate the lookup table relating CO and patient activity during intrinsic heartbeats;

FIG. 5 is a flowchart illustrating an exemplary procedure for adjusting the paired rate to meet physiological demand based on patient activity for use with the technique of FIG. 2 that exploits the lookup table of FIG. 4;

FIG. 6 illustrates exemplary signal components processed by the control method of FIG. 5 that exploits the lookup table relating CO and patient activity to set the paired pacing rate;

FIG. 7 is a simplified, partly cutaway view, illustrating the device of FIG. 1 along with at set of leads implanted into the heart of the patient; and

FIG. 8 is a functional block diagram of the pacer/ICD of FIG. 7, illustrating basic circuit elements that provide cardioversion, defibrillation and/or pacing stimulation in the heart and particularly illustrating components for controlling the techniques of FIGS. 2-6.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable System and Method

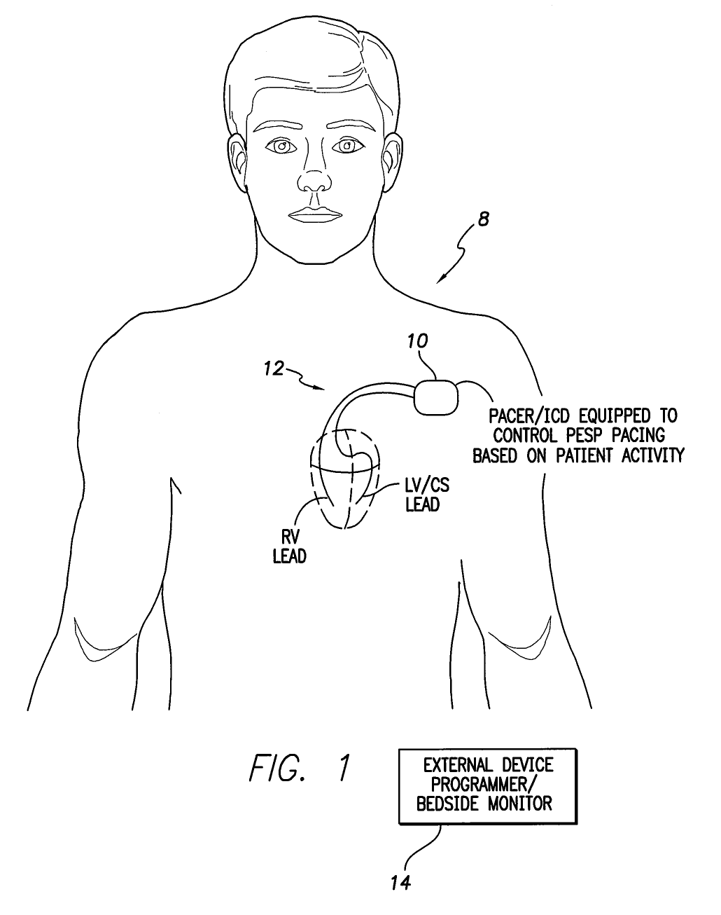

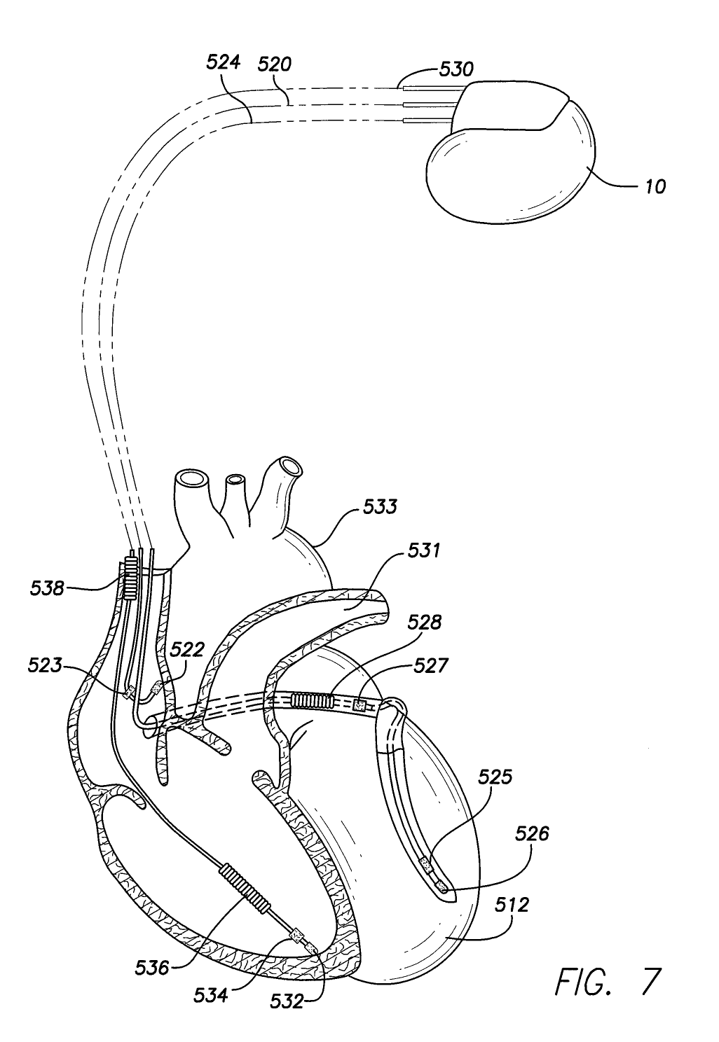

FIG. 1 illustrates an implantable medical system 8 capable of delivering PESP via paired pacing while adjusting the paired pacing rate based on patient activity. In this example, the implantable medical system includes a pacer/ICD 10 or other cardiac stimulation device (such as a CRT device) equipped with a set of cardiac sensing/pacing leads 12 implanted on or within the heart of the patient, including at least an RV lead and an LV lead implanted via the coronary sinus (CS) for biventricular pacing. In FIG. 1, a stylized representation of the leads is set forth. A more accurate and complete illustration of the leads is provided within FIG. 7, discussed below. In the exemplary embodiments described herein, the paired pacing is delivered using the LV and RV leads in accordance with biventricular pacing techniques but other pacing vectors can be used.

The pacer/ICD is programmed using an external programming device 14 under clinician control. Programming commands can specify, for example, the circumstances under which paired pacing should be activated within the patient, the time delay between the pulses of each pair and the amplitudes of the pulses. At other times, the pacer/ICD may be in communication with a beside monitor or other diagnostic device such as a personal advisory module (PAM) that receives and displays data from the pacer/ICD, such as diagnostic data representative of the efficacy of paired pacing. In some embodiments, the bedside monitor is directly networked with a centralized computing system, such as the HouseCall™ system or the Merlin@home/Merlin.Net systems of St. Jude Medical, which can relay diagnostic information to the clinician.

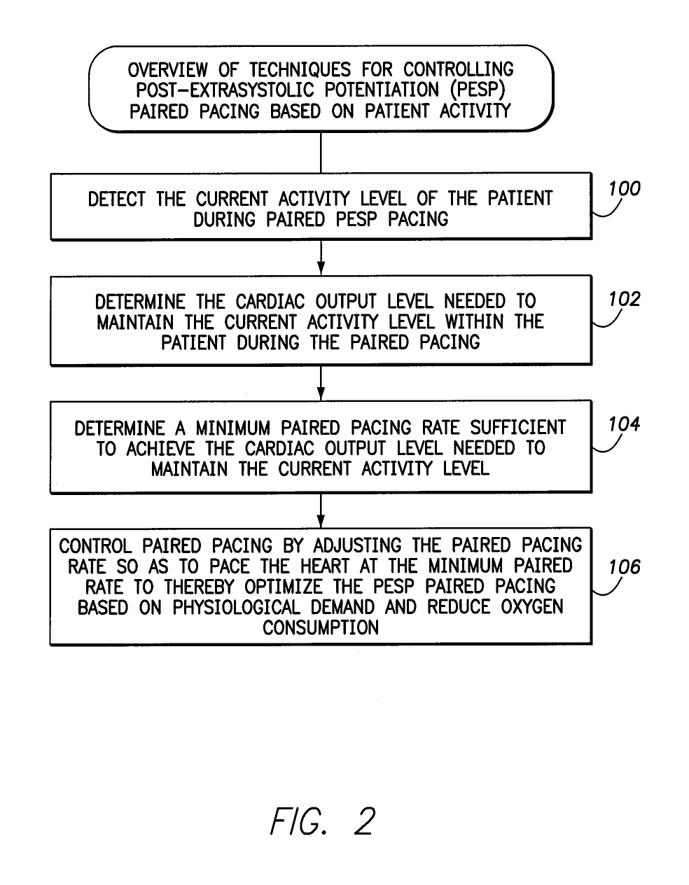

FIG. 2 broadly summarizes techniques employed by the pacer/ICD of FIG. 1 (or other suitably-equipped systems) for controlling paired pacing based on patient activity. Briefly, beginning at step 100, the pacer/ICD detects the current activity level of the patient during on-going paired PESP pacing, i.e. while paired pacing is being delivered to the heart of the patient. At step 102, the pacer/ICD determines the cardiac output (CO) level needed to maintain the current activity level within the patient during the paired pacing. As will be described in more detail below, this determination may be made with reference to lookup tables that relate CO to activity levels within the patient. The lookup tables may be generated during a preliminary calibration/initialization procedure. At step 104, the pacer/ICD determines a paired pacing rate sufficient to achieve the CO level needed to maintain the current activity level (i.e. needed to meet the physiological demands due to that activity.) This pacing rate may be ascertained by dividing the determined CO level by the current stroke volume (SV) within the patient, estimated based on changes in cardiogenic/intracardiac impedance (Z). At step 106, the pacer/ICD then controls paired pacing by adjusting the paired pacing rate so as to pace the heart at the paired rate determined at step 104 to thereby (substantially) optimize the PESP paired pacing based on physiological demand and to reduce oxygen consumption (relative to paired pacing techniques that would instead pace at a higher rate), or to achieve other advantageous benefits.

### Preliminary Calibration/Initialization Procedure

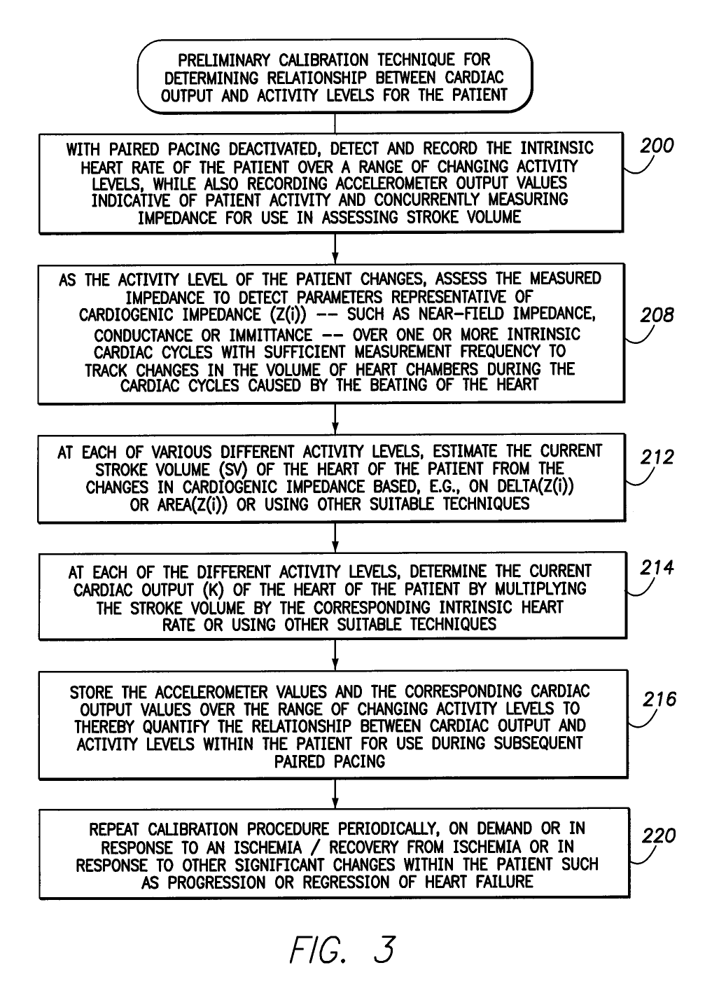

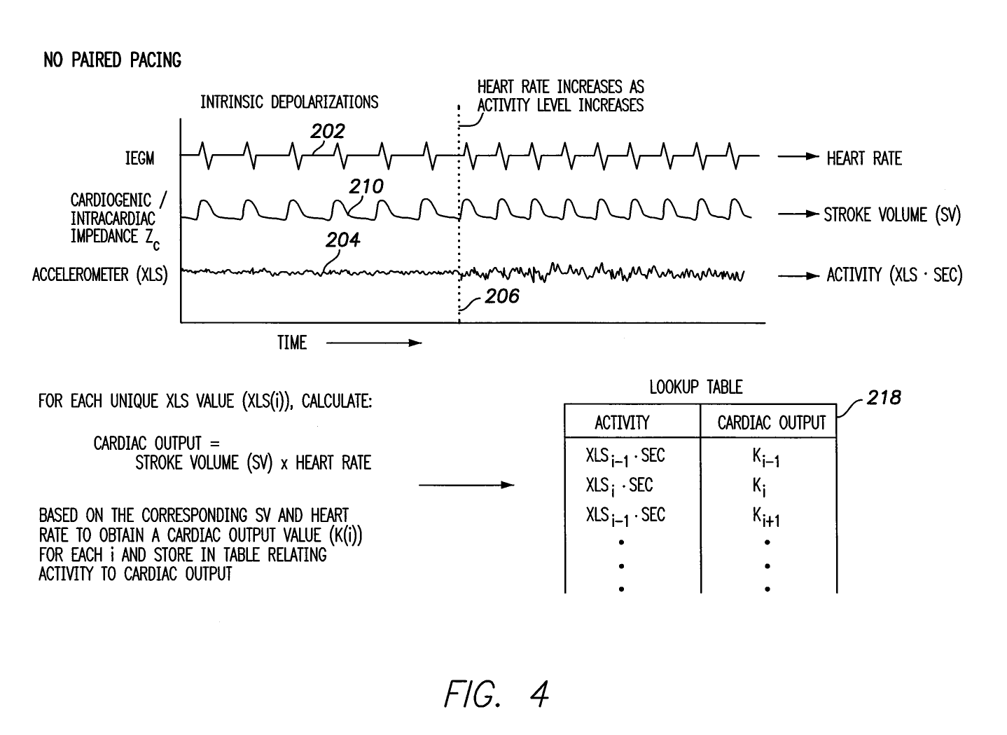

FIGS. 3 and 4 illustrate techniques that may employed by a device programmer, other external systems, or by the pacer/ICD itself for determining the relationship between CO and patient activity for the particular patient in which the pacer/ICD is implanted, i.e. for determining the minimum CO needed to meet various levels of patient activity within the patient. Data quantify the relationship between CO and patient activity levels may be generated and stored in a lookup table (or functional equivalent) for use during subsequent paired pacing to control the pacing rate to meet the physiological demands of the patient.

Beginning at step 200 of FIG. 3, with paired pacing deactivated, the system detects and records the intrinsic heart rate of the patient over a range of changing activity levels, while also recording accelerometer output values indicative of patient activity and concurrently measuring impedance for use in assessing stroke volume. For example, during a post-implant followup session with the clinician, the patient may be instructed to exercise on a treadmill, with the speed adjusted so as to vary the level of patient activity through a range of activity levels from relatively mild to relatively more strenuous. FIG. 4 illustrates the intrinsic heart rate by way of IEGM trace 202, along with exemplary accelerometer output values 204. In this example, the patient activity level increases at time 206, resulting in accelerometer signals of greater magnitude, and also a higher heart rate.

At step 208 of FIG. 3, as the activity level of the patient changes, the system assesses the impedance values measured at step 200 to detect parameters representative of cardiogenic/intracardiac impedance (Z(i))—such as near-field impedance, conductance or immittance—over one or more intrinsic cardiac cycles with sufficient measurement frequency to track changes in the volume of heart chambers during the cardiac cycles caused by the beating of the heart. Trace 210 of FIG. 4 illustrates the cardiogenic/intracardiac impedance signal, which varies in magnitude over each heartbeat by an amount sufficient to allow SV to be estimated. Preferably, the cardiogenic/intracardiac impedance signals are detected using the leads of the device itself based on suitable impedance detection pulses or waveforms. A particularly effective tri-phasic impedance detection pulse for use in detecting impedance is described in U.S. patent application Ser. No. 11/558,194 of Panescu et al., filed Nov. 9, 2006, entitled “Closed-Loop Adaptive Adjustment of Pacing Therapy based on Cardiogenic Impedance Signals Detected by an Implantable Medical Device.”

If an external system is performing the calibration procedure of FIG. 3, the cardiogenic impedance data may be transmitted via telemetry from the pacer/ICD to the external system along with IEGM and accelerometer data. If the pacer/ICD is performing the calibration procedure, then the data is merely stored and processed internally.

At step 212, at each of the different activity levels, the system estimates the current SV of the heart of the patient from the changes in cardiogenic impedance detected within in individual heartbeats based, e.g., on delta(Z(i)) or area(Z(i)) or using other suitable techniques. Otherwise conventional techniques may be used to estimate SV from impedance. See, for example, impedance-based techniques discussed in U.S. Published Patent Application 2011/0046691 of Bjorling et al., entitled “Implantable Heart Stimulator determining Left Ventricular Systolic Pressure.”

At step 214, at each of the different activity levels, the system determines the current CO (abbreviated “K”) of the heart of the patient by multiplying the SV estimated at step 212 by the corresponding intrinsic heart rate detected at step 200 to thereby generate a set of values of K as a function of activity (XLS) throughout a range of patient activity levels. For example, for each unique XLS value (XLS(i)), the system calculates: CARDIAC OUTPUT=STROKE VOLUME (SV)×HEART RATE based on the corresponding SV and heart rate to obtain a CO value (K(i)) for each i. The resulting CO values may be regarded as the minimum CO needed by the patient to maintain the corresponding level of activity. As can be appreciated, for a given level of activity, a set of CO values obtained over time at that activity level can be averaged together to provide a more robust estimate of the true CO required by the patient at that particular level of activity.

At step 216, the system then stores the accelerometer values XLS (or, preferably, XLS·SEC, where XLS·SEC represents the accelerometer output signals summed or integrated over time such as over one cardiac cycle) and the corresponding CO values (K(i)) over the range of changing activity levels to thereby quantify the relationship between CO and activity levels within the patient for use during subsequent paired pacing. Within FIG. 4, an exemplary lookup table 218 is illustrated. As already explained, rather than using a lookup table, functional equivalents may instead be used, such as linear equations that relate CO to activity using coefficients determined via linear regression or other suitable techniques.

At step 220, the system can repeat the calibration/initialization procedure to update or replace the values within the lookup table. This may be done periodically (such as every month or two), on-demand (based on clinician control) or automatically in response to detection of an episode of ischemia within the patient (or recovery from ischemia) or in response to other significant changes within the patient, such as progression or regression of heart failure. A variety of techniques may be employed to detect ischemia in the patient. See, for example, U.S. Patent Application 2011/0004111 of Gill et al., entitled “Ischemia Detection using Intra-Cardiac Signals”; U.S. Pat. No. 6,108,577 to Benser, entitled “Method and Apparatus for Detecting Changes in Electrocardiogram Signals;” and U.S. Pat. No. 7,610,086 to Ke et al., entitled “System and Method for Detecting Cardiac Ischemia in Real-Time using a Pattern Classifier Implemented within an Implanted Medical Device.” A variety of techniques may be employed to track heart failure. See, for example, U.S. Pat. No. 7,676,260 to Koh, entitled “Implantable Cardiac Stimulation Device that Monitors Progression and Regression of Heart Disease Responsive to Differences in Averaged Electrograms and Method” and U.S. Pat. No. 7,171,271 to Koh et al., entitled “System and Method for Evaluating Heart Failure using an Implantable Medical Device based on Heart Rate During Patient Activity.”

Hence, FIGS. 3 and 4 set forth exemplary techniques for initializing the system by determining CO and activity values for populating the lookup table. In these examples, CO is estimated from SV and heart rate, where SV is estimated from cardiogenic impedance. Other techniques may be used, additionally or alternatively, to estimate or directly detect SV or to estimate or directly detect CO. See, for example, techniques discussed in: U.S. Pat. No. 7,139,609 to Min et al., entitled “System and Method for Monitoring Cardiac Function via Cardiac Sounds using an Implantable Cardiac Stimulation Device”; U.S. Pat. No. 7,654,964 to Kroll et al. entitled “System and Method for Detecting Arterial Blood Pressure based on Aortic Electrical Resistance using an Implantable Medical Device”; U.S. Pat. No. 7,925,347 to Bornzin, entitled “Assessment of Cardiac Output by Implantable Medical Device”; and U.S. Pat. No. 7,632,235 to Karicherla et al., entitled “System and Method for Measuring Cardiac Output via Thermal Dilution using an Implantable Medical Device with an External Ultrasound Power Delivery System.”

### Activity-Based Control of Paired Pacing Rate

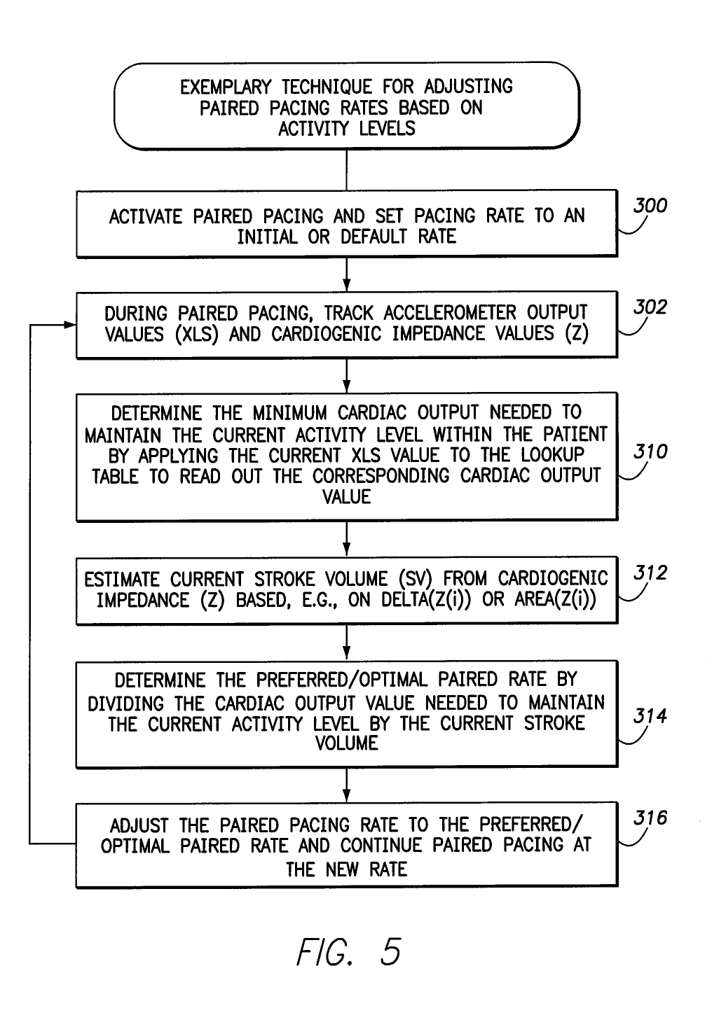

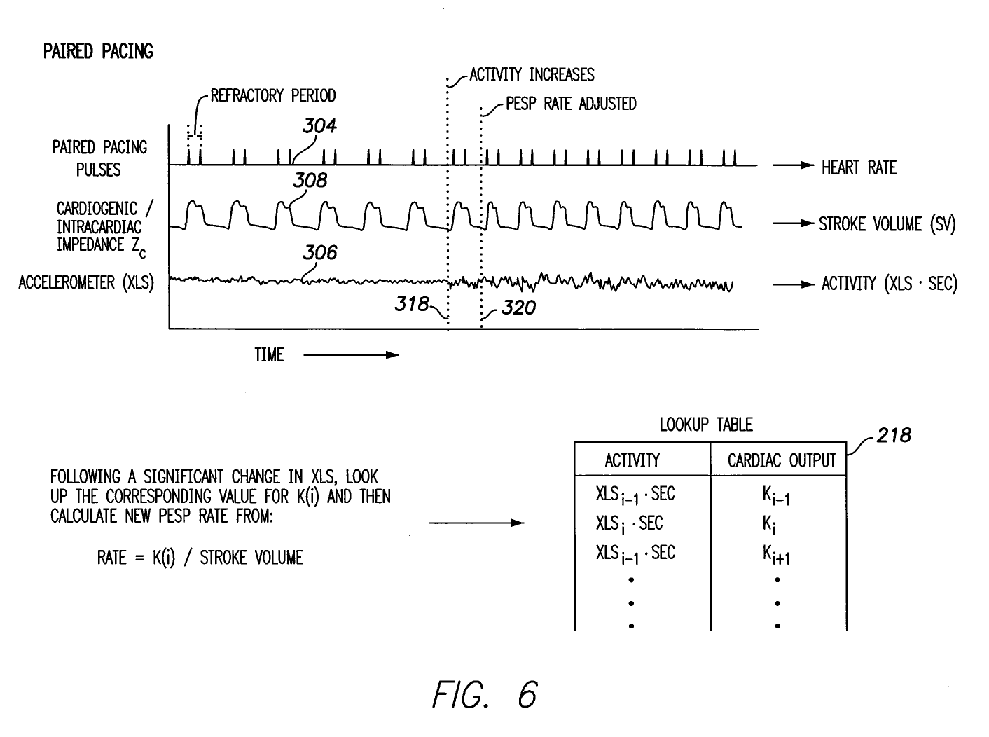

FIGS. 5 and 6 illustrate techniques that may be employed by the pacer/ICD for automatically adjusting the paired pacing rate based on patient activity to optimize the PESP pacing based on physiological demand and to reduce oxygen consumption or to achieve other advantageous benefits. These techniques utilize the lookup table data generated by the calibration/initialization techniques of FIGS. 3 and 4.

Beginning at step 300 of FIG. 5, the pacer/ICD activates paired pacing and sets the paired pacing rate to an initial or default rate. This initial paired pacing rate may be the rate used by otherwise conventional paired pacing systems and may be set, for example, based on an examination of the current intrinsic heart rate and other factors. Note that with paired pacing, a primary stimulation pulse is delivered to the heart of the patient using the leads wherein the primary pulse has a pulse amplitude/duration sufficient to depolarize and contract myocardial tissue. The subsequent absolute and relative refractory periods are tracked using otherwise conventional techniques. Then a secondary (paired) stimulation pulse is delivered during (or just beyond) the relative refractory period. The secondary stimulation pulse has a pulse amplitude/duration sufficient to depolarize myocardial tissue without triggering contraction (i.e. the pulse is configured to achieve PESP.) The pulse amplitude and width of the primary and secondary PESP pulses may be set using otherwise conventional techniques. PESP therapy and related techniques are also discussed in: U.S. Pat. No. 7,184,833; U.S. Pat. No. 5,213,098; U.S. Pat. No. 7,289,850; U.S. Patent Application 2007/0250122; U.S. Patent Application 2006/0149184; U.S. Patent Application 2006/0247698 and U.S. Patent Application 2007/0250122.

At step 302, during paired pacing, the pacer/ICD tracks accelerometer output values (XLS) and cardiogenic/intracardiac impedance values (Z). FIG. 6 illustrates paired pacing by way of IEGM 304, along with exemplary accelerometer output values 306 and cardiogenic/intracardiac impedance values 308. The total refractory period associated with the primary pulse of the first pair of pulses is also shown. As can be seen, the second pulse of the pair is delivered within the refractory period to achieve PESP.

At step 310 of FIG. 5, the pacer/ICD determines the minimum CO needed to maintain the current activity level within the patient by applying the current XLS value (or a summed version thereof such as XLS·SEC) to lookup table 218 to read out the corresponding cardiac output value (K), which may be regarded as representing the minimum CO value needed to sustain the current activity level. This value may also be regarded as a “target” CO value to be achieved within the patient via a change in the paired pacing rate. At step 312, the pacer/ICD estimates current SV from cardiogenic impedance (Z) based, e.g., on delta(Z(i)) or area(Z(i)) or using other suitable techniques. At step 314, the pacer/ICD determines the preferred/optimal paired pacing rate by dividing the CO value needed to maintain the current activity level (obtained from the lookup table at step 310) by the current SV (estimated at step 312.) At step 316, the pacer/ICD adjusts the paired pacing rate (if needed) to the preferred/optimal paired rate and continues paired pacing at the new rate. In this regard, if the current paired rate is sufficient to meet the physiological demands of the patient based on the current activity level, then the paired pacing rate need not be increased (though it might be adjusted downwardly if deemed to be too high.) On the other hand, if the current paired rate is not sufficient to meet the physiological demands of the patient, then the paired pacing rate is increased to the preferred/optimal rate to increase CO to meet the physiological demands brought on by the current level of activity. Steps 302-316 may be repeated every few cardiac cycles (or potentially every cardiac cycle) to repeatedly update the paired pacing rate to respond to changing activity levels within the patient. Preferably, it is repeated at least once every ten cardiac cycles or at least once every ten seconds. Alternatively, respiration cycles can be tracked and exploited during the adjustment of the paired pacing rate since respiration modulates stroke volume.

Note that in the example of FIG. 6, patient activity increases at time 318, triggering an increase in the magnitude of the accelerometer signals (XLS). In response to this increase, the new XLS value (or a summed version thereof) is applied to the lookup table to readout a new value for K. The new value for K is divided by the current estimate of SV to determine a new paired pacing rate, which is applied at time 320 to increase CO within the patient to meet the higher physiological demands arising due to the increased level of activity. Thereafter, if the activity level subsequently decreases, the new lower XLS value is applied to the table to read out a new lower value for K, which yields a new (lower) optimal pacing rate, triggering a decrease in the rate at which paired pacing is delivered to the patient.

Hence, FIGS. 5 and 6 set forth exemplary techniques for controlling paired pacing rates based on patient activity. In these examples, SV is again estimated from cardiogenic impedance. Other techniques may be used, additionally or alternatively, to estimate or directly detect SV. See, for example, techniques discussed in the patent documents cited above.

Although primarily described with respect to examples having a pacer/ICD, other implantable medical devices may be equipped to exploit the techniques described herein, such as standalone CRT devices or CRT-D devices (i.e. a CRT device also equipped to deliver defibrillation shocks.) CRT and related therapies are discussed in, for example, U.S. Pat. No. 6,643,546 to Mathis, et al., entitled “Multi-Electrode Apparatus and Method for Treatment of Congestive Heart Failure”; U.S. Pat. No. 6,628,988 to Kramer, et al., entitled “Apparatus and Method for Reversal of Myocardial Remodeling with Electrical Stimulation”; and U.S. Pat. No. 6,512,952 to Stahmann, et al., entitled “Method and Apparatus for Maintaining Synchronized Pacing”. See, also, U.S. Patent Application No. 2008/0306567 of Park et al., entitled “System and Method for Improving CRT Response and Identifying Potential Non-Responders to CRT Therapy” and U.S. Patent Application No. 2007/0179390 of Schecter, entitled “Global Cardiac Performance.” The techniques described herein may also be applicable to systems equipped for multi-site LV (MSLV) pacing, such as systems using quad-pole leads or the like. See, for example, techniques described in U.S. Patent Application 2011/0022112 of Min, entitled “Systems and Methods for Determining Ventricular Pacing Sites for use with Multi-Pole Leads.”

It should be understood that the “optimal” pacing rates obtained using the techniques described herein are not necessarily absolutely optimal in a given quantifiable or mathematical sense. What constitutes “optimal” depends on the criteria used for judging the resulting performance, which can be subjective in the minds of some clinicians. The paired pacing rates determined by the techniques described herein represent, at least, “preferred” pacing rates. Clinicians may choose to adjust or alter the rates for particular patients, at their discretion.

For the sake of completeness, an exemplary pacer/ICD will now be described, which includes components for performing or controlling the functions and steps already described.

### Exemplary Pacer/ICD

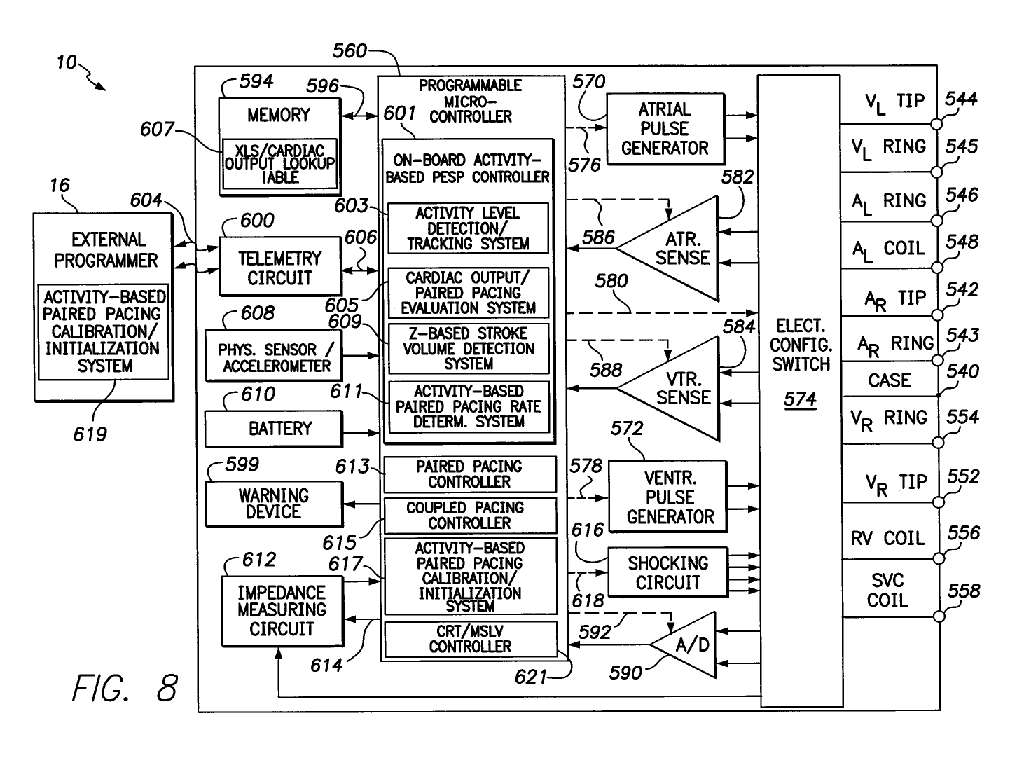

With reference to FIGS. 7 and 8, a description of an exemplary pacer/ICD will now be provided. FIG. 7 provides a simplified block diagram of the pacer/ICD, which is a dual-chamber stimulation device capable of treating both fast and slow arrhythmias with stimulation therapy, including cardioversion, defibrillation, and pacing stimulation, and also capable of paired pacing to achieve PESP. To provide atrial chamber pacing stimulation and sensing, pacer/ICD 10 is shown in electrical communication with a heart 512 by way of a left atrial lead 520 having an atrial tip electrode 522 and an atrial ring electrode 523 implanted in the atrial appendage. Pacer/ICD 10 is also in electrical communication with the heart by way of a right ventricular lead 530 having, in this embodiment, a ventricular tip electrode 532, a right ventricular ring electrode 534, a right ventricular (RV) coil electrode 536, and a superior vena cava (SVC) coil electrode 538. Typically, the right ventricular lead 530 is transvenously inserted into the heart so as to place the RV coil electrode 536 in the right ventricular apex, and the SVC coil electrode 538 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a CS lead 524 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary CS lead 524 is designed to receive atrial and ventricular cardiac signals and to deliver left ventricular pacing therapy using at least a left ventricular (LV) tip electrode 526 and a LV ring electrode 525, left atrial pacing therapy using at least a left atrial (LA) ring electrode 527, and shocking therapy using at least a LA coil electrode 528. With this configuration, biventricular pacing can be performed. Although only three leads are shown in FIG. 7, it should also be understood that additional leads (with one or more pacing, sensing and/or shocking electrodes) might be used and/or additional electrodes might be provided on the leads already shown.

A simplified block diagram of internal components of pacer/ICD 10 is shown in FIG. 8. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 540 for pacer/ICD 10, shown schematically in FIG. 8, is often referred to as the “can”, “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544, 545, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the RA tip electrode 522 and a right atrial ring terminal (A_{R} RING) 543 adapted for connection to the RA ring electrode 523. To achieve left chamber sensing, pacing and shocking, the connector includes at least a left ventricular tip terminal (V_{L} TIP) 544, a left ventricular ring terminal (V_{L} RING) 545, a left atrial ring terminal (A_{L} RING) 546, and a left atrial shocking terminal (A_{L} COIL) 548, which are adapted for connection to the LV tip electrode 526, the LV ring electrode 525, the LA ring electrode 527, and the LA coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (V_{R} COIL) 556, and an SVC shocking terminal (SVC COIL) 558, which are adapted for connection to the RV tip electrode 532, RV ring electrode 534, the RV coil electrode 536, and the SVC coil electrode 538, respectively.

At the core of pacer/ICD 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 8, an atrial pulse generator 570 and a ventricular pulse generator 572 generate pacing stimulation pulses for delivery by the right atrial lead 520, the right ventricular lead 530, and/or the CS lead 524 via an electrode configuration switch 574. It is understood that in order to provide stimulation therapy in each of the four chambers of the heart, the atrial and ventricular pulse generators 570, 572, may include dedicated, independent pulse generators, multiplexed pulse generators or shared pulse generators. The pulse generators 570, 572, are controlled by the microcontroller 560 via appropriate control signals 576, 578, respectively, to trigger or inhibit the stimulation pulses.

The microcontroller 560 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art.

Atrial sensing circuits 582 and ventricular sensing circuits 584 may also be selectively coupled to the right atrial lead 520, CS lead 524, and the right ventricular lead 530, through the switch 574 for detecting the presence of cardiac activity in each of the four chambers of the heart. Accordingly, the atrial and ventricular sensing circuits 582, 584 may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit 582, 584 preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits 582, 584 are connected to the microcontroller 560 which, in turn, are able to trigger or inhibit the atrial and ventricular pulse generators 570, 572, respectively, in a demand fashion in response to the absence or presence of cardiac activity in the appropriate chambers of the heart.

For arrhythmia detection, pacer/ICD 10 utilizes the atrial and ventricular sensing circuits 582, 584 to sense cardiac signals to determine whether a rhythm is physiologic or pathologic. As used in this section, “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 590. The data acquisition system 590 is configured to acquire intracardiac electrogram signals, convert the raw analog data into a digital signal, and store the digital signals for later processing and/or telemetric transmission to an external device 16. The data acquisition system 590 is coupled to the right atrial lead 520, the CS lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of pacer/ICD 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 594 through a telemetry circuit 600 in telemetric communication with the external device 16, such as a programmer, transtelephonic transceiver or a diagnostic system analyzer. The telemetry circuit 600 is activated by the microcontroller by a control signal 606. The telemetry circuit 600 advantageously allows intracardiac electrograms and status information relating to the operation of pacer/ICD 10 (as contained in the microcontroller 560 or memory 594) to be sent to the external device 16 through an established communication link 604. Pacer/ICD 10 further includes an accelerometer or other physiologic sensor or sensors 608, sometimes referred to as a “rate-responsive” sensor because it is typically used to adjust pacing stimulation rate according to the exercise state of the patient.

However, physiological sensor(s) 608 can be equipped to sense any of a variety of cardiomechanical parameters, such as heart sounds, systemic pressure, etc. As can be appreciated, at least some these sensors may be mounted outside of the housing of the device and, in many cases, will be mounted to the leads of the device. Moreover, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV delay, V-V delay, etc.) at which the atrial and ventricular pulse generators, 570 and 572, generate stimulation pulses. While shown as being included within pacer/ICD 10, it is to be understood that the physiologic sensor 608 may also be external to pacer/ICD 10, yet still be implanted within or carried by the patient. A common type of rate responsive sensor is an activity sensor incorporating an accelerometer or a piezoelectric crystal and/or a 3D-accelerometer capable of determining the posture within a given patient, which is mounted within the housing 540 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 610, which provides operating power to all of the circuits shown in FIG. 8. The battery 610 may vary depending on the capabilities of pacer/ICD 10. If the system only provides low voltage therapy, a lithium iodine or lithium copper fluoride cell typically may be utilized. For pacer/ICD 10, which employs shocking therapy, the battery 610 should be capable of operating at low current drains for long periods, and then be capable of providing high-current pulses (for capacitor charging) when the patient requires a shock pulse. The battery 610 should also have a predictable discharge characteristic so that elective replacement time can be detected. Accordingly, appropriate batteries are employed.

As further shown in FIG. 8, pacer/ICD 10 is shown as having an impedance measuring circuit 612, which is enabled by the microcontroller 560 via a control signal 614. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; measuring cardiogenic/intracardiac impedance, and detecting the opening of heart valves, etc. The impedance measuring circuit 612 is advantageously coupled to the switch 674 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-10 joules) or high energy (11 to 40 joules or more), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 6-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

Insofar as PESP pacing is concerned, the microcontroller includes on on-board activity-based PESP controller 601 operative to detect a current activity level of the patient during paired pacing; determine a cardiac output level needed to maintain the current activity level of the patient during paired pacing; and determine a paired pacing rate sufficient to achieve the cardiac output level. To this end, the controller includes an activity level detection/tracking system 603, which may operate in conjunction with accelerometer 608, to assess the current level of activity of the patient. A cardiac output/paired pacing evaluation system 605 determines the cardiac output level needed to maintain the current activity level during paired pacing with reference, for example, to an XLS/cardiac output lookup table 607 (or functional equivalent) within memory 594. A Z-based stroke volume detection system 609 estimates the current SV for the heart of the patient based, for example, on impedance signals measured using circuit 612. An activity-based paired pacing rate determination system 611 determines the paired pacing rate needed to meet the CO level determined by system 605 by, for example, dividing CO by SV to yield a preferred/optimal paired pacing rate. A paired pacing controller 613 controls the delivery of paired pacing via the various pulse generators. Additionally, for the sake of completeness, a coupled pacing controller 615 is also shown.

To initialize or calibrate the data stored in lookup table 607, an on-board activity-based paired pacing calibration/initialization system 617 is provided. Additionally or alternatively, these functions may be performed by an external activity-based paired pacing calibration/initialization system 619, which transmits the lookup table data (or functional equivalents) to the device for storage within device memory. A CRT/MSLV controller 621 is also shown within the microcontroller of the device to control CRT and/or MSLV pacing. For multi-site left ventricular (MSLV) pacing, additional terminals may be required to accommodate additional multi-site stimulation electrodes of a multi-polar LV lead, such as a quad-pole lead.

Any diagnostic data pertinent to PESP pacing can be stored in memory 594 for eventual transmission to an external system. In the event any warnings are needed, such as warnings pertaining to PESP pacing, such warnings can be delivered using an onboard warning device, which may be, e.g., a vibrational device or a “tickle” voltage warning device.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A method for use with an implantable cardiac stimulation device equipped to deliver paired pacing within a patient, the method comprising:
detecting a current activity level of the patient during paired pacing;
determining a cardiac output level needed to maintain the current activity level of the patient during paired pacing;
determining a paired pacing rate sufficient to achieve the cardiac output level; and
delivering paired pacing at the determined pacing rate.

2. The method of claim 1 wherein the cardiac output level needed to maintain the current activity level is determined based on a predetermined relationship between activity level and cardiac output for the patient.

3. The method of claim 2 wherein the predetermined relationship between activity level and cardiac output is represented by a lookup table relating activity levels to corresponding cardiac output levels for the patient over a range of activity levels.

4. The method of claim 3 wherein the current patient activity level is detected using an accelerometer and wherein the lookup table relates accelerometer output values to corresponding cardiac output levels for the patient over a range of accelerometer output values.

5. The method of claim 1 wherein determining a paired pacing rate sufficient to achieve the cardiac output level includes:
determining a current stroke volume for the patient during paired pacing; and
determining a paired pacing rate sufficient to achieve the cardiac output level based on the current stroke volume of the patient.

6. The method of claim 5 wherein determining the current stroke volume for the patient includes:
detecting parameters representative of cardiogenic impedance over at least one cardiac cycle; and
estimating stroke volume from the parameters representative of cardiogenic impedance.

7. The method of claim 6 wherein the values representative of cardiogenic impedance include one or more of impedance, admittance, conductance and immittance.

8. The method of claim 5 wherein the paired pacing rate sufficient to achieve the cardiac output level is determined by dividing the cardiac output level by the stroke volume.

9. The method of claim 1 wherein determining the paired pacing rate is performed to determine the minimum rate sufficient to achieve the cardiac output level.

10. The method of claim 1 wherein at least some of the steps of detecting the current activity level of the patient, determining the cardiac output level needed to maintain the current activity level, and determining the paired pacing rate are repeated periodically to update the paired pacing rate.

11. The method of claim 1 wherein at least some of the steps of detecting the current activity level of the patient, determining the cardiac output level needed to maintain the current activity level, and determining the paired pacing rate are repeated on-demand to update the paired pacing rate.

12. The method of claim 1 further including a preliminary procedure performed without pair pacing for determining cardiac output levels needed to maintain current activity levels within the patient over at range of activity levels.

13. The method of claim 12 wherein the preliminary procedure includes:
for a range of patient activity levels, determining corresponding cardiac output levels for the patient during intrinsic cardiac cycles; and
generating and storing a representation of a relationship between activity level and cardiac output within the patient over the range of activity levels for subsequent use during paired pacing.

14. The method of claim 13 wherein patient activity is determined during the preliminary procedure using an accelerometer and the relationship between activity level and cardiac output is represented by a lookup table relating accelerometer output values to corresponding cardiac output levels over the range of activity levels.

15. The method of claim 14 wherein determining corresponding cardiac output levels for the patient during the preliminary procedure includes:
detecting an intrinsic heart rate;
detecting parameters representative of cardiogenic impedance over at least one intrinsic cardiac cycle;
estimating stroke volume from the parameters representative of cardiogenic impedance;
determining cardiac output by multiplying stroke volume by the intrinsic heart rate.

16. The method of claim 1 wherein all of the steps are performed by the implantable medical device.

17. The method of claim 1 wherein at least some of the steps are performed by an external device in communication with the implantable medical device.

18. A system for use with an implantable cardiac stimulation device equipped to deliver paired pacing within a patient, the system comprising:
an activity level detection system operative to detect a current activity level of the patient during paired pacing;
a cardiac output/paired pacing evaluation system operative to determine a cardiac output level needed to maintain the current activity level during paired pacing;
an activity-based paired pacing rate determination system operative to determine a paired pacing rate sufficient to achieve the cardiac output level needed to maintain the current activity level; and
a paired pacing controller operative to control the delivery of paired pacing at the determined pacing rate.

19. A system for use with an implantable cardiac stimulation device equipped to deliver paired pacing within a patient, the system comprising:
means for detecting a current activity level of the patient during paired pacing;
means for determining a cardiac output level needed to maintain the current activity level during paired pacing;
means for determining a paired pacing rate sufficient to achieve the cardiac output level of the patient; and
means for controlling the delivery of paired pacing at the determined pacing rate.
